(19) United States
(12) Patent Application Publication
(10) Pub. No.: US 2024/0298891 A1
(43) Pub. Date: Sep. 12, 2024

(54) FOLDABLE DENTAL SCOPE

(71) Applicant: Get-Grin Inc., Airmont, NY (US)

(72) Inventors: Yarden EILAT-BLOCH, Haifa (IL); Carmi RAZ, Gizo (IL)

(21) Appl. No.: 18/669,880

(22) Filed: May 21, 2024

Related U.S. Application Data
(63) Continuation of application No. PCT/US2022/050881, filed on Nov. 23, 2022.
(60) Provisional application No. 63/283,058, filed on Nov. 24, 2021.

(51) Int. Cl. A61B 1/24; A61B 1/00

(52) U.S. Cl. CPC A61B 1/24; A61B 1/00128

(57) ABSTRACT

Described herein are various configurations of a foldable dental scope and methods and systems to use such a scope. In an aspect, provided herein is a foldable dental scope that is configured to transform between an unfolded configuration and a folded configuration, wherein in said unfolded configuration, the dental scope comprises a sheet comprising one or more distinct regions separated by scoring, and wherein in said folded configuration, said sheet forms an intraoral adapter comprising a viewing channel configured to define a field of view of an intraoral region of a subject's mouth.

### CROSS REFERENCE TO RELATED APPLICATIONS

This application is a continuation of International Application No. PCT/US2022/050881 filed Nov. 23, 2022, which claims benefit of U.S. Provisional Application No. 63/283,058 filed Nov. 24, 2021, which is incorporated herein by reference in its entirety.

### TECHNICAL FIELD

The systems and methods described herein are related to remote dental assessment, and more specifically, a foldable dental scope that can be folded and used by a user to capture intraoral images or videos.

### BACKGROUND

Dental professionals may treat and monitor a patient's dental condition based on in-person visits. Treatment and monitoring of a patient's dental condition may require a patient to schedule multiple in-person visits to a dentist. The quality of treatment and the accuracy of monitoring may vary depending on how often and how consistently a patient sees a dentist. In some cases, suboptimal treatment outcomes may result if a patient is unable or unwilling to schedule regular visits to a dentist.

### SUMMARY

Recognized herein is a need for remote dental monitoring solutions to allow dental patients to receive high quality dental care, without requiring a dental professional to be physically present with the patient. Some dental professionals may use conventional teledentistry solutions to accommodate patients' needs and schedules. However, such conventional teledentistry solutions may provide inadequate levels of supervision. Further, such conventional teledentistry solutions may be limited by an inaccurate or insufficient monitoring of a patient's dental condition based on one or more photos or videos taken by the patient, if the photos do not adequately capture various intraoral features.

The present disclosure provides systems and methods for remote dental assessment or monitoring. As used herein, remote dental assessment or monitoring may refer to remote onsite assessments conducted by remote personal and may refer to the acquisition of one or more intraoral videos and/or intraoral images that can assess remotely by remote computing device, or to be send to different location and assess in there. The systems and methods disclosed herein may provide a convenient solution and user experience for dental patients to capture one or more intraoral videos or images using a mobile device such as a smartphone. The methods and systems disclosed herein may provide dentists with a detailed analysis of the patient's dental condition based on one or more full arch scans captured remotely by the patient.

The present disclosure provides systems and methods that can be provided to the user or a patient in an envelope, and the user or a patient can assemble the foldable scope on site. In addition, the present disclosure can provide the option for home printing and/or cutting of a foldable dental scope described herein. The present disclosure provides systems and methods for folding a dental scope out of available materials on site to provide remote dental assessment or monitoring.

In an aspect, provided herein is a foldable dental scope that is configured to transform between an unfolded configuration and a folded configuration, wherein in the unfolded configuration, the dental scope comprises a sheet comprising one or more distinct regions separated by scoring, and wherein in the folded configuration, the sheet forms an intraoral adapter comprising a viewing channel configured to define a field of view of an intraoral region of a subject's mouth.

In some cases, in the folded configuration, the intraoral adapter comprises an elongated housing, and wherein the viewing channel is located between a proximal end and a distal end of the elongated housing.

In some cases, the proximal end of the elongated housing is configured to couple the intraoral adapter to the subject's mouth.

In some cases, the distal end of the elongated housing is configured to couple to a mobile imaging device.

In some cases, the distal end of the elongated housing foldable comprises an attachment element configured to couple the intraoral adapter to a mobile imaging device.

In some cases, the attachment element comprises a sticker, an adhesive, Velcro, or a magnet, or any combination thereof.

In some cases, the attachment element comprises one or more tabs.

In some cases, the attachment element comprises a strip.

In some cases, the strip is sufficiently long to wrap around the mobile imaging device.

In some cases, the strip comprises a fastening element configured to secure a first portion of the strip to a second portion of the strip.

In some cases, the fastening element comprises a sticker, an adhesive, Velcro, or a magnet, or any combination thereof.

In some cases, the strip comprises a fastening element configured to secure a first portion of the strip to the mobile imaging device.

In some cases, the fastening element comprises a sticker, an adhesive, Velcro, or a magnet, or any combination thereof.

In some cases, the mobile imaging device is a mobile phone.

In some cases, the elongated housing comprises one or more walls.

In some cases, the one or more walls of the elongated housing in the folded configuration correspond to the one or more discrete regions in the unfolded configuration.

In some cases, the one or more distinct regions each comprise a first end and a second end.

In some cases, the first ends of the one or more distinct regions are curved.

In some cases, the first ends of the one or more distinct regions are concave or convex.

In some cases, the first ends of the one or more discrete regions in the unfolded

configuration correspond to the proximal end of the elongated housing in the folded configuration.

In some cases, the proximal end of the elongated housing is configured to couple the intraoral adapter to the subject's mouth.

In some cases, the first ends of the one or more discrete regions in the unfolded configuration alternate between a concave and convex shape.

In some cases, the concave shape is configured to contact a top and bottom lip of the subject's mouth.

In some cases, the concave shape is configured to hold a lip of the subject outside of the field of view of the intraoral region of the subject's mouth.

In some cases, the convex shape is configured to contact a side of the subject's mouth.

In some cases, the convex shape is configured to hold a cheek of the subject outside of the field of view of the intraoral region of the subject's mouth.

In some cases, one or more of the second ends are configured to be folded outward, and wherein in the folded configuration, the one or more of the second ends extend outward from the viewing channel.

In some cases, the second ends of the one or more distinct regions are straight.

In some cases, the second ends of the one or more discrete regions in the unfolded configuration correspond to the distal end of the elongated housing in the folded configuration.

In some cases, the distal end of the elongated housing is configured to couple to a mobile imaging device.

In some cases, a first distinct region of the one or more distinct regions comprises a notch or slit, and wherein a second distinct region of the one or more distinct regions comprises a tab or rim configured to be inserted in the notch or slit to prevent the folded configuration from unfolding.

In some cases, when in the unfolded configuration, the foldable dental scope is flat.

In some cases, when in the unfolded configuration, the foldable dental scope is configured to fit into an envelope for shipping to a user.

In some cases, the foldable dental scope comprises plastic.

In some cases, the foldable dental scope comprises paper.

In some cases, the foldable dental scope comprises carboard.

In some cases, the foldable dental scope comprises silicon.

In some cases, the foldable dental scope comprises metal.

In some cases, the scoring facilitates folding of the sheet.

In some cases, the scoring comprises indentations or marks in the sheet.

In some cases, in the folded configuration, the viewing channel has a cross-sectional shape.

In some cases, the cross-sectional shape is a polygon.

In some cases, the polygon has four sides.

In some cases, the cross-sectional shape is a rectangle.

In some cases, the cross-sectional shape is a trapezoid.

In some cases, the one or more distinct regions are substantially trapezoidal in shape.

In another aspect, provided herein is a rollable dental scope that is configured to transform between an unrolled configuration and a rolled configuration, wherein in the unrolled configuration, the dental scope comprises a sheet, and wherein in the rolled configuration, the sheet has been rolled to form an intraoral adapter comprising a viewing channel configured to define a field of view of an intraoral region of a subject's mouth.

In some cases, in the rolled configuration, the intraoral adapter comprises an elongated housing, and wherein the viewing channel is located between a proximal end and a distal end of the elongated housing.

In some cases, the proximal end of the elongated housing is configured to couple the intraoral adapter to the subject's mouth.

In some cases, the distal end of the elongated housing is configured to couple to a mobile imaging device.

In some cases, the distal end of the elongated housing rollable comprises an attachment element configured to couple the intraoral adapter to a mobile imaging device.

In some cases, the attachment element comprises a sticker, an adhesive, Velcro, or a magnet, or any combination thereof.

In some cases, the attachment element comprises one or more tabs.

In some cases, the attachment element comprises a strip.

In some cases, the strip is sufficiently long to wrap around the mobile imaging device.

In some cases, the strip comprises a fastening element configured to secure a first portion of the strip to a second portion of the strip.

In some cases, the fastening element comprises a sticker, an adhesive, Velcro, or a magnet, or any combination thereof.

In some cases, the strip comprises a fastening element configured to secure a first portion of the strip to the mobile imaging device.

In some cases, the fastening element comprises a sticker, an adhesive, Velcro, or a magnet, or any combination thereof.

In some cases, the mobile imaging device is a mobile phone.

In some cases, the sheet comprises a first end and a second end.

In some cases, the first end is curved.

In some cases, the first end comprises alternating convex and concave sections.

In some cases, the first end of the sheet in the unrolled configuration corresponds to the proximal end of the elongated housing in the rolled configuration.

In some cases, the proximal end of the elongated housing is configured to couple the intraoral adapter to the subject's mouth.

In some cases, the concave sections are configured to contact a top and bottom lip of the subject's mouth.

In some cases, the concave sections are configured to hold a lip of the subject outside of the field of view of the intraoral region of the subject's mouth.

In some cases, the convex sections are configured to contact a side of the subject's mouth.

In some cases, the convex sections are configured to hold a cheek of the subject outside of the field of view of the intraoral region of the subject's mouth.

In some cases, the first end is configured to be folded outward, and wherein in the rolled configuration, the first extends outward from the viewing channel.

In some cases, the second end is straight.

In some cases, the second end of the sheet in the unrolled configuration corresponds to the distal end of the elongated housing in the rolled configuration.

In some cases, the distal end of the elongated housing is configured to couple to a mobile imaging device.

In some cases, a first region of the sheet comprises a notch or slit, and wherein a second region of the sheet comprises a tab or rim configured to be inserted in the notch or slit to prevent the rolled configuration from unrolling.

In some cases, when in the unrolled configuration, the rollable dental scope is flat.

In some cases, when in the unrolled configuration, the rollable dental scope is configured to fit into an envelope for shipping to a user.

In some cases, the rollable dental scope comprises plastic.

In some cases, the rollable dental scope comprises paper.

In some cases, the rollable dental scope comprises carboard.

In some cases, the rollable dental scope comprises silicon.

In some cases, the rollable dental scope comprises metal.

In some cases, in the rolled configuration, the viewing channel has a cross-sectional shape.

In some cases, the cross-sectional shape is a circle.

In some cases, the cross-sectional shape is an oval.

In some cases, the cross-sectional shape is an ellipse.

In another aspect, provided herein is a method for intraoral imaging, comprising: providing a foldable dental scope in an unfolded configuration, wherein the foldable dental scope is configured to transform between the unfolded configuration and a folded configuration; transforming the foldable dental scope into the folded configuration, wherein in the folded configuration, the foldable dental scope comprises a viewing channel that defines a field of view of an intraoral region of a subject's mouth; coupling a first end of the foldable dental scope to a mobile imaging device; placing a second end of the foldable dental scope in the subject's mouth; and using the mobile imaging device, capturing an image or video of the subject's intraoral region through the viewing channel.

In some cases, the image or video is captured by a user, and wherein the image or video is provided to a dental health professional for assessment.

In some cases, the assessment comprises assessment of a dental condition.

In some cases, the dental condition comprises (i) a movement of one or more teeth of the subject, (ii) an accumulation of plaque on one or more teeth of the subject, (iii) a change in a color or a structure of one or more teeth of the subject, (iv) a change in a color or a structure of a tissue adjacent to one or more teeth of the subject, (v) a presence or lack of presence of one or more cavities, or (vi) a condition of the subject's gums, tongue, uvula, airway, tonsils, or any oral tissue.

In some cases, the assessment is used to generate or update a dental treatment plan.

In some cases, the dental treatment plan is a cosmetic treatment plan.

In some cases, the assessment is used to assess the subject's actual progress in relation to a dental treatment plan.

In some cases, the method is performed with the aid of instructions detailing how to transform the unfolded configuration to the folded configuration.

In some cases, the image or video comprises a 3D dental scan of the subject's mouth.

In some cases, transforming the foldable dental scope into the folded configuration comprises inserting a tab located on a first portion of the foldable dental scope into a notch or slit located on a second portion of the foldable dental scope, thereby preventing the folded configuration from unfolding.

In another aspect, provided herein is a method to attach a foldable dental scope to a mobile imaging device, comprising: providing a foldable dental scope in an unfolded configuration, wherein the foldable dental scope is configured to transform between the unfolded configuration and a folded configuration; transforming the foldable dental scope into the folded configuration, wherein in the folded configuration, the foldable dental scope comprises an attachment element configured to couple the intraoral adapter to a mobile imaging device; and using the attachment element, coupling the foldable dental scope to the mobile imaging device.

In some cases, the attachment element comprises a sticker, an adhesive, Velcro, or a magnet, or any combination thereof.

In some cases, the attachment element comprises one or more tabs.

In some cases, the attachment element comprises a strip.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the method and system of the present invention involves performing or completing selected tasks or steps manually, automatically, or a combination thereof. Moreover, according to actual instrumentation and equipment of preferred embodiments of methods, apparatus and systems of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1A schematically illustrates an example of a foldable dental scope in an unfolded configuration, in accordance with some embodiments.

FIG. 1B schematically illustrates an example of a strip that can be used to couple a foldable dental scope to a mobile imaging device, in accordance with some embodiments.

FIG. 2A schematically illustrates an example of a foldable dental scope in an unfolded configuration, in accordance with some embodiments.

FIG. 2B schematically illustrates an example of an attachment mechanism that can be used to couple a foldable dental scope to a mobile imaging device, in accordance with some embodiments.

FIG. 3 schematically illustrates an example of a foldable dental scope and a strip, in accordance with some embodiments.

FIG. 4 schematically illustrates an example of a foldable dental scope in a folded configuration with four tabs configured to couple a foldable dental scope to a mobile imaging device, in accordance with some embodiments.

FIG. 5 schematically illustrates an example of a foldable dental scope in a folded configuratoin, in accordance with some embodiments.

FIG. 6A schematically illustrates an example of a rollable dental scope foldable cut sheet in an unrolled configuration, in accordance with some embodiments.

FIG. 6B schematically illustrate an example of a strip that can be used to couple a rollable dental scope to a mobile imaging device, in accordance with some embodiments.

FIG. 6C schematically illustrates an example of a rollable dental scope in a rolled configuration, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “a,” “an,” and “the,” as used herein, generally refer to singular and plural references unless the context clearly dictates otherwise.

### Overview

In an aspect, the present disclosure provides systems and methods for intraoral assessment. As used herein, intraoral assessment may refer assessment of the teeth and or teeth condition, or any other intraoral tissue assessment or condition. The methods and systems disclosed herein may provide a convenient solution and user experience for dental patients to capture one or more intraoral videos or images using a mobile device such as a smartphone. The methods and systems disclosed herein may provide dentists and orthodontists with a detailed analysis of the patient's dental condition based on one image or more of a tooth and or full arch scans captured remotely by the patient.

In an aspect, the present disclosure provides solution to fold a dental scope out of a sheet. In some cases, the sheet can be flat. As used herein the term “dental scope” or “dental adapter” refer to a device that can be attached to a camera or mobile phone and enable capturing intraoral images or videos of a patient or a user. An example and possible uses can be seen on U.S. patent application Ser. No. 17/336,997. The systems and methods of the present disclosure may be implemented using a software application that is configured to enable a dental patient to capture videos and/or images of intraoral regions. The software application may be used by a user or a subject (e.g., a dental patient) in conjunction with a mobile device to remotely monitor a dental condition of the subject. The dental condition may comprise a development, a growth, a movement, an appearance, a condition, a physical arrangement, a position, and/or an orientation of the subject's teeth.

The mouth and dental assessment may be used to enable remote monitoring. As used herein, remote monitoring may refer to monitoring intraoral condition of a patient that is performed at one or more locations remote from the patient. For example, a dentist or a medical specialist may monitor the dental anatomy or dental condition in a first location that is different than a second location where the patient is located. The first location and the second location may be separated by a distance spanning at least 1 meter, 1 kilometer, 10 kilometers, 100 kilometers, 1000 kilometers, or more.

The remote monitoring may be performed by assessing a dental condition of the subject using one or more dental scan captured by the subject when the patient is located remotely from the dentist or a dental office. In some cases, the remote monitoring may be performed in real-time such that a dentist is able to assess the dental condition when a subject uses a mobile device to acquire one or more dental scan of one or more intraoral regions in the patient's mouth. The remote monitoring may be performed using equipment, hardware, and/or software that is not physically located at a dental office.

### Device for Intraoral Assessment

In an aspect, the present disclosure provides a foldable sheet comprising four attached trapezoid-shaped regions with alternating concave and convex curved bottoms; and a rim extending outward from the trapezoid-shaped regions. In some cases, the foldable sheet can be folded into a trapezoid-shaped box with two opposites open sides. The narrow open side may have a straight edge, and the wider open side may have a curved edge with a rim that folds outward.

In some embodiments the foldable sheet further comprises at least two rims expending from the straight upper side of at least two trapezoid-shaped shapes.

In some embodiments, the rims comprise an attachment element.

In some embodiments, the foldable sheet can be attached to a mobile phone using the attachment element.

In some embodiments, the attachment element is a sicker, Velcro, or a magnetic element.

In some embodiments the foldable sheet further comprises a notch or slit on one or more of the trapezoid-shaped regions.

In some embodiments, a first side of a strip can be inserted into the notch or slit.

In some embodiments, a second side of a strip may comprise adhesive, Velcro or magnetic element.

In some embodiments, the foldable sheet is made from more then four trapezoid-shaped regions.

In some embodiments, the foldable sheet can be attached to a mobile phone using the strip.

In some embodiments, the foldable sheet can be made of stiff and foldable material.

In some embodiments, the stiff and foldable material is plastic, cardboard, paper, silicon, metal, or any suitable material.

In some embodiments, the foldable sheet comprises folding lines and the folding lines are marked or dented. In some cases, the foldable sheet comprises scoring in between said distinct regions. The scoring may be used to facilitate folding of said foldable sheet along said scoring.

In some embodiments, one side of the foldable dental scope has an additional rim extending on all the sides that can fold over the opposite side when folded.

In some embodiments, the foldable sheet, attachment means, and instructions for folding or unfolding the scope can be provided to a user in a flat envelope.

The present disclosure provides a system for assessing the mouth condition of a user, comprising: a mobile phone with a camera and a processor anda foldable dental scope, wherein the system configuration allows the user to perform assessments.

In some embodiments the user can fold the foldable sheet into a trapezoid-shaped box. In some cases, a narrow straight edge opening of the trapezoid-shaped box is attached to a mobile phone in a manner that surrounds the mobile phone camera.

In some embodiments, the user is a remote user.

The present disclosure provides a method for assessing a mouth condition of a remote user, comprising: providing a foldable scope; providing a mobile phone comprising a camera and a processor; instructing the user on how to fold and attach the foldable scope to the mobile phone; instructing the user to place a curve side of the folded trapezoid-shaped box into the mouth, wherein the curve side of the folded trapezoid-shaped box is coupled to a user's mouth, wherein the user's lips touch the outer side of the folded trapezoid-shaped box; instructing the user to obtain an image and/or a video of the inner mouth with the mobile phone; and assessing the image and or video provided by the user.

In some embodiments the assessment comprises: medical, pathology or appearance condition of a tooth, gum, tongue, uvula, airway, glands or any Oral tissue of a user.

In some embodiments, the method is used for remote consultation

In some embodiments, the method is used for public survey.

In some embodiments, the method is used to provide medical or cosmetic treatment to a user.

In some embodiments, the method is used for 3D mapping of the oral cavity and teeth.

In some embodiments, the user can be further instructed on how to attach the trapezoid-shaped box to a mobile imaging device, like a phone, unfold the folded trapezoid-shaped box into a flat sheet, and hygienically treat the sheet for storage.

In another aspect, the present disclosure provides a method to attacha foldable dental adapter or dental scope to a mobile phone comprising:
providing an attachment element located on a proximal side of the dental adapter or dental scope; providing an attachment element attached to a phone and surrounding the mobile phone camera. wherein the attachment of the dental adapter or dental scope to the mobile phone is by sticker. Velcro or magnets.

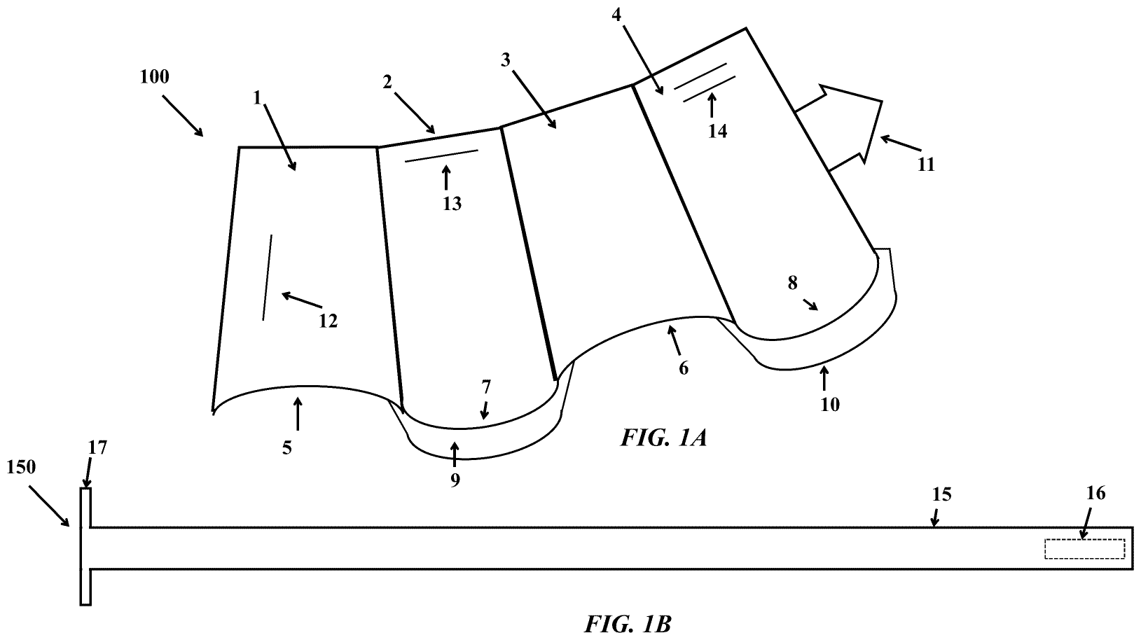

FIG. 1A schematically illustrates an example of a foldable sheet. The cut sheet 100, comprises four trapezoid-shaped pieces 1,2,3,4. The 1,3 trapezoid-shaped pieces have concave bottoms, 5 and 6. The 2 and 4 numbered trapezoid-shaped pieces have convex bottoms, 7 and 8. In some cases, additional convex rims 9 and 10 are connected to the convex bottom, 7 and 8. The foldable sheet 100 can be provided to a user or a patient in a cut form or as a full sheet that the shape can be cut from. The sheet 100 can include an additional rim 11 on the side of trapezoid-shaped 4 piece. The sheet 100 can include additional slits or notches 12, 13 and 14. Slice 12 can be used to insert the rim or tab 11 when the sheet is folded to keep the cut sheet in the fold position. Slit 13 can be the insertion slit or notch for the strip 150. Slits 14 and 15 can be used to tighten the strip 150. In some embodiments, additional convex rims 9 and 10 that are connected to the convex bottom, 7 and 8 can be folded out when folding the sheet 100 to form a box shape that can be used as dental scope.

FIG. 1B schematically illustrate an example of a strip that can be used with the foldable sheet. The strip 150 can be used to attach the foldable sheet 100 to a mobile imaging device. The strip 150 may have an extension 17 on one end and an attachment element 16 on the other end. The strip 15 may be sufficiently long enough to secure the assembled sheet 100 to a smart phone or a tablet. Is some embodiments, the strip 150 is made from the same material as the foldable sheet. In some embodiments, the strip 150 is made from a flexible material. The strip 150 can be inserted into slit 13 in FIG. 1 from the inner side. The extension 17 may prevent the strip from sliding. The strip can be used to cover the smart phone and can be inserted into slit 14 and back outward from the second slit and then be secured to itself via the attachment element 16. According to some embodiments, the attachment element 16 is a sticker, an adhesive, a Velcro, or a magnetic element. The attachment element 16 can be attached to the strip.

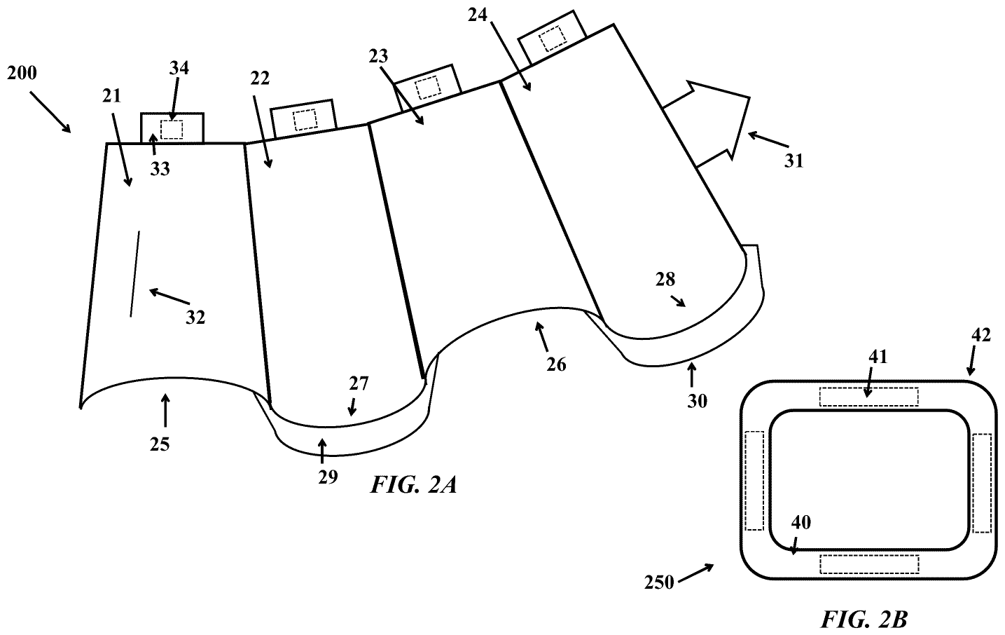

FIG. 2A schematically illustrates an example of a foldable sheet. The cut sheet 200, may comprise four trapezoid-shaped pieces 21,22,23,24. The 21,23 trapezoid-shaped pieces may have concave bottoms, 25 and 26. The 22 and 24 numbered trapezoid-shaped pieces may have convex bottoms, 27 and 28. In some cases, additional convex rims 29 and 30 are connected to the convex bottoms, 27 and 28. The sheet 200 can be provided to a user or a patient in a cut form or as a full sheet that the shape can be cut from. The sheet 200 can include an additional rim 31 on the side of trapezoid-shaped 24 piece. The cut sheet 200 can include an additional slit 32. Slit 32 can be used to insert the rim or tab 31 when the cut sheet is folded to keep the cut sheet in the fold position. At least two additional rims, 33 can be part of the cut sheet 200. The additional rims 33 can include an attachment element 34. According to some embodiments, the attachment element 34 is a sticker, a Velcro, or a magnetic element and can be used to attach the folded sheet 200 to a mobile imaging device.

FIG. 2B schematically illustrates an example of a mobile phone sticker 250. The mobile phone sticker 250 can have a square shape 40 and a sticker 42 that can be used to attach the mobile phone sticker 40 to a mobile phone. The mobile phone sticker 250 can include one or more attachment elements 41. According to some embodiments, the attachment element 41 is a sticker, Velcro, or a magnetic element and can be use when attaching the folded sheet 200 in a folded configuration to a mobile phone.

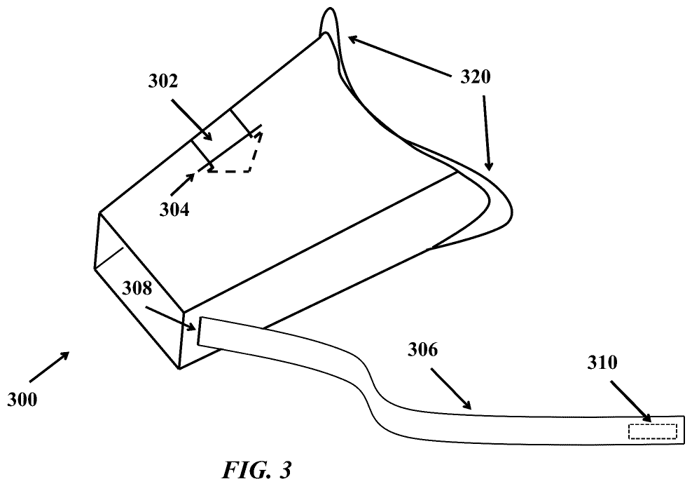

FIG. 3 illustrates an example of a foldable sheet 100 folded into dental scope 300. The rim 302 can be inserted into slice 304 to hold the sheet in the dental scop shape. In addition, the rims 320 are folded outward. The rims 320 can be placed in a user's mouth and may help keep a cheek of a user out of a field of view of the scope. Strip 306 can be inserted through slit 308 while the attachment element 310 is used to secure the folded cut sheet 300 with the strip 306 to a cell phone.

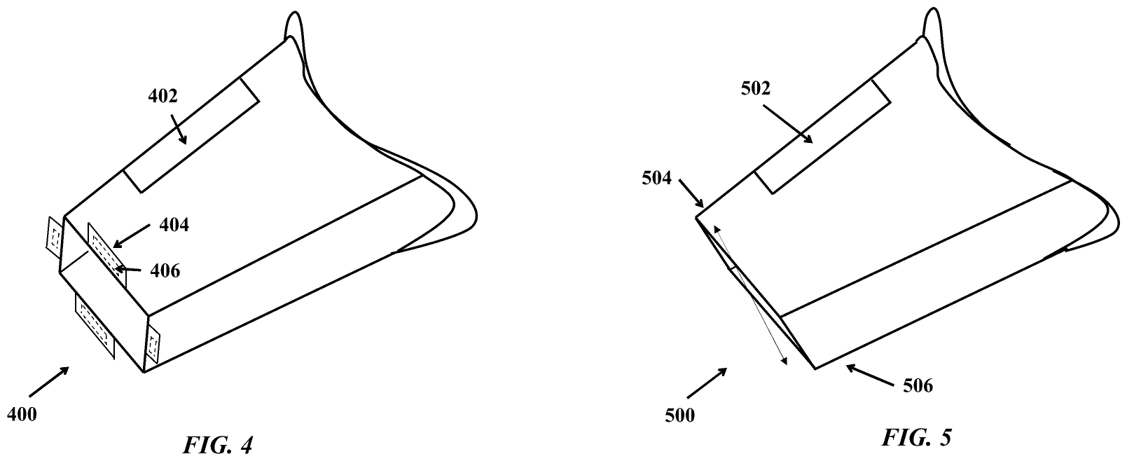

FIG. 4 illustrates an example of a sheet 200 folded into dental scope 400. The rim 402 can be attached to the outer end to hold the sheet in a dental scope shape. The dental scope can be attached to a mobile imaging device using a sticker, Velcro, or a magnetic element. In addition, the additional convex rims can be folded outward. At least two additional rims 404 can be part of the sheet 200. The additional rims 404 can include an attachment element 406. According to some embodiments, the attachment element 406 is a sticker, Velcro, or a magnetic element. The attachment element 406 can be used to attach the folded sheet 400 in the shape of dental scope to the mobile phone.

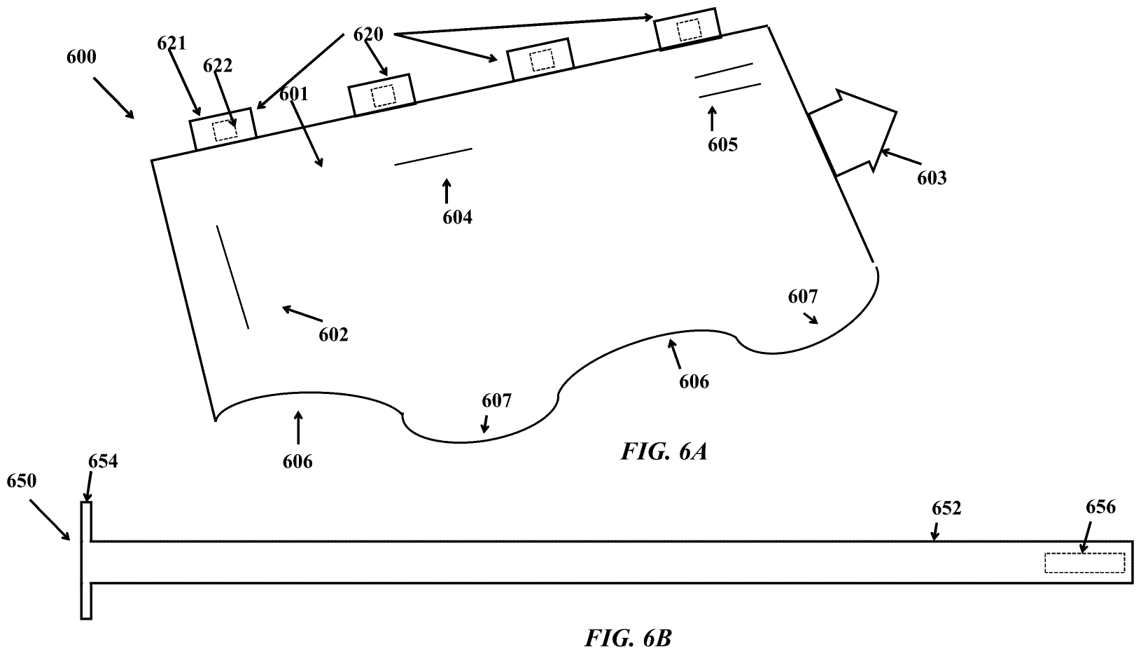

FIG. 5 illustrates an example of a sheet 500 folded into dental scope 500. The rim 502 can be glued into place, and the dental scope 500 can be provided to the user or the patient in a collapsed shape in a flat envelope. The user or the patient can push the dental scope 500 from the folded corners 504 and 506 towards each other to form an uncollapsed dental scope 500 in a desired shape. Additional convex rims can be folded outward before use.

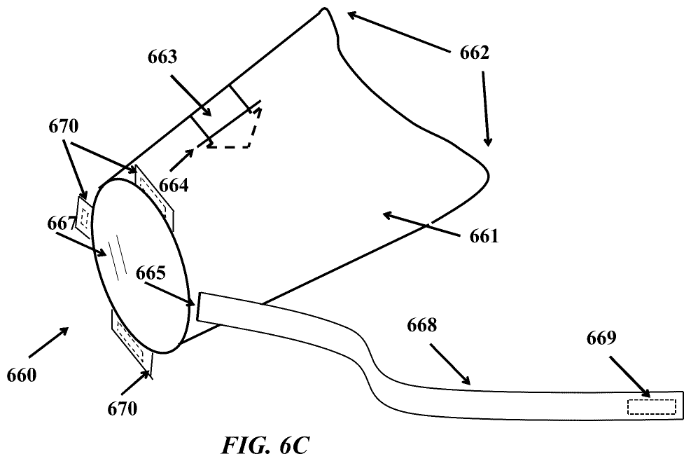

FIG. 6A schematically illustrates an example of a foldable sheet. The sheet 600 may comprise one piece 601. The shape 601 can comprise a concave 606 bottom and a convex 607 bottom. The sheet 600 can be provided to a user or a patient in a cut form, or as a full sheet that the shape can be cut from. The sheet 600 can include an additional rim or tab 603 on a side of the piece. The sheet 600 can include slits 602, 604 and 605. Slit 602 can be used to insert the rim or tab 603 when the sheet is folded to keep the cut sheet in the folded position. Slit 604 can be the insertion slice for the strip 650 (FIG. 6B). Slices 605 can be used to tighten the strip 650. In some embodiments, the sheet 600 can include at least two additional rims 620. The additional rims 620 can include an attachment element 622. According to some embodiments, the attachment element 622 is a sticker, a Velcro, or a magnetic element. The attachment element 622 can be used when attaching the folded cut sheet 600 in the shape of dental scope to a mobile imaging device.

FIG. 6B schematically illustrates an example of a strip that can be used with the foldable sheet. The strip 650 can have an extension 654 on one end and an attachment element 656 on the other end. The strip 652 may be sufficiently long to secure the assembled sheet 600 to a smart phone or a tablet. In some embodiments, the strip 650 is made from the same material as the sheet. In some embodiments, the strip 650 is made from a flexible material. The strip 650 can be inserted into slit 604 from FIG. 6A from the inner side. The extension 654 may prevent the strip from sliding. The strip 654 can be used to cover the smart phone. The strip 654 may be inserted into slit 605 and then inserted back outward from the second slice and be secured to itself via the attachment element 656. According to some embodiments, the attachment element 656 is a sticker, Velcro, or a magnetic element. In some cases, the attachment element 656 is attached to the strip.

FIG. 6C illustrates an example of a sheet 600 folded or rolled into dental scope 660. The rim or tab 663 can be inserted into slit 664 to hold the sheet in the dental scope shape. Strip 668 can be inserted through slit 665. The attachment element 669 can be used to secure the folded sheet 660 with the strip 668 to a mobile imaging device. In some embodiments, at least two additional rims 670 can be part of the sheet 660. The additional rims 670 can include an attachment element. According to some embodiments, the attachment element 670 can be a sticker, Velcro, or a magnetic element. The rims 670 can be used to attach the folded sheet 660 in the shape of dental scope to a mobile phone.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense.

Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A foldable dental scope that is configured to transform between an unfolded configuration and a folded configuration,
wherein in said unfolded configuration, the dental scope comprises a sheet comprising one or more distinct regions separated by scoring, and
wherein in said folded configuration, said sheet forms an intraoral adapter comprising a viewing channel configured to define a field of view of an intraoral region of a subject's mouth.

2. The foldable dental scope of claim 1, wherein in said folded configuration, said intraoral adapter comprises an elongated housing, and wherein said viewing channel is located between a proximal end and a distal end of said elongated housing.

3. The foldable dental scope of claim 2, wherein said proximal end of said elongated housing is configured to couple the intraoral adapter to the subject's mouth.

4. The foldable dental scope of claim 2, wherein said distal end of said elongated housing foldable comprises an attachment element configured to couple said intraoral adapter to a mobile imaging device.

5. The foldable dental scope of claim 4, wherein said attachment element comprises a sticker, an adhesive, Velcro, a strap, or a magnet, or any combination thereof.

6. The foldable dental scope of claim 1, wherein a first distinct region of said one or more distinct regions comprises a notch or slit, and wherein a second distinct region of said one or more distinct regions comprises a tab or rim configured to be inserted in said notch or slit to prevent said folded configuration from unfolding.

7. A rollable dental scope that is configured to transform between an unrolled configuration and a rolled configuration, wherein in said unrolled configuration, the dental scope comprises a sheet, and wherein in said rolled configuration, said sheet has been rolled to form an intraoral adapter comprising a viewing channel configured to define a field of view of an intraoral region of a subject's mouth.

8. The rollable dental scope of claim 7, wherein in said rolled configuration, said intraoral adapter comprises an elongated housing, and wherein said viewing channel is located between a proximal end and a distal end of said elongated housing.

9. The rollable dental scope of claim 8, wherein said proximal end of said elongated housing is configured to couple the intraoral adapter to the subject's mouth.

10. The rollable dental scope of claim 8, wherein said distal end of said elongated housing rollable comprises an attachment element configured to couple said intraoral adapter to a mobile imaging device.

11. The rollable dental scope of claim 10, wherein said attachment element comprises a sticker, an adhesive, Velcro, a strap, or a magnet, or any combination thereof.

12. The rollable dental scope of claim 7, wherein a first region of said sheet comprises a notch or slit, and wherein a second region of said sheet comprises a tab or rim configured to be inserted in said notch or slit to prevent said rolled configuration from unrolling.

13. A method for intraoral imaging, comprising:
(a) providing a foldable dental scope in an unfolded configuration, wherein said foldable dental scope is configured to transform between said unfolded configuration and a folded configuration;
(b) transforming said foldable dental scope into said folded configuration, wherein in said folded configuration, said foldable dental scope comprises a viewing channel that defines a field of view of an intraoral region of a subject's mouth;
(c) coupling a first end of said foldable dental scope to a mobile imaging device;
(d) placing a second end of said foldable dental scope in said subject's mouth; and
(e) using said mobile imaging device, capturing an image or video of said subject's intraoral region through said viewing channel.

14. The method of claim 13, wherein said image or video is captured by a user, and wherein said image or video is provided to a dental health professional for assessment.

15. The method of claim 14, wherein said assessment comprises assessment of a dental condition.

16. The method of claim 15, wherein said dental condition comprises (i) a movement of one or more teeth of the subject, (ii) an accumulation of plaque on one or more teeth of the subject, (iii) a change in a color or a structure of one or more teeth of the subject, (iv) a change in a color or a structure of a tissue adjacent to one or more teeth of the subject, (v) a presence or lack of presence of one or more cavities, or (vi) a condition of said subject's gums, tongue, uvula, airway, tonsils, or any oral tissue.

17. The method of claim 14, wherein said assessment is used to generate or update a dental treatment plan.

18. The method of claim 17, wherein said dental treatment plan is a cosmetic treatment plan.

19. The method of claim 14, wherein said assessment is used to assess said subject's actual progress in relation to a dental treatment plan.

20. The method of claim 13, wherein (b) is performed with the aid of instructions detailing how to transform said unfolded configuration to said folded configuration.
